(19) United States
(12) Patent Application Publication
(10) Pub. No.: US 2005/0053658 A1
(43) Pub. Date: Mar. 10, 2005

(54) Extended release systems for macrolide antibiotics

(76) Inventors: Gopi M. Venkatesh, Vandalia, OH (US); Nehal H. Vyas, Vandalia, OH (US); Ken Kangyi Qian, Beavercreek, OH (US)

Correspondence Address: THOMPSON HINE L.L.P., 2000 COURTHOUSE PLAZA , N.E., 10 WEST SECOND STREET, DAYTON, OH, 45402, US

(21) Appl. No.: 10/658,078

(22) Filed: Sep. 9, 2003

(51) Int. Cl. A61K 31/7048; A61K 9/22

(52) U.S. Cl. 424/468; 514/29

(57) ABSTRACT

A unit dosage form, such as a tablet or the like for delivering poorly water soluble macrolide antibiotics such as Clarithromycin into the body in an extended release fashion, is designed to release the active ingredient primarily by tablet erosion though the tablet composition does not comprise any dissolution rate controlling agent, a polymer or esters of fatty acids. Such a drug delivery system provides a plasma concentration—time profile suitable for once a day oral administration.

### CROSS REFERENCE

This application claims the benefit of U.S. Provisional Application, Atty. Docket No. 451194-092-P filed Jul. 2, 2003.

### TECHNICAL FIELD

The present invention is related to an extended release solid pharmaceutical composition in the tablet form comprising no dissolution rate controlling polymer, or an ester of a fatty acid, for once-a-day oral administration of a poorly water soluble macrolide antibiotic, such as clarithromycin, which is indicated for the treatment of microbial infection. The dosage form described in this application is based on the drug release mechanism primarily by tablet erosion.

### BACKGROUND OF THE INVENTION

Many therapeutic agents are most effective when made available at a constant rate. The absorption of therapeutic agents thus made available generally results in desired plasma concentrations leading to maximum efficacy and minimum toxic side effects. Much effort has been devoted to developing sophisticated drug delivery systems, such as osmotic devices, providing drug release at a constant rate for oral application. However, there are instances, as described in this invention, where a tablet formulation of a poorly water soluble drug comprising pharmaceutically acceptable excipients which are normally used for the preparation of immediate release tablet formulations, such as a binder, diluent(s), lubricant and glidant, provides an in vitro extended release profile by tablet erosion and maintains appropriate blood level of the drug upon once a day administration. Since the tablet formulation of clarithromycin comprises no dissolution rate controlling excipients, the tablet size is small and provides for the convenience of administering a single tablet of 1000 mg instead of two units of 500 mg each (example: Biaxin XL 500 mg).

Macrolide antibiotics, such as erythromycin and its derivatives, are known for their antibacterial activity against a number of organisms and are generally administered 2-3 times a day as immediate release dosage forms. These antibiotics have a bitter metallic taste, which often results in poor compliance of the regimen. In order to address the frequency and duration of the administration and/or the adverse effects related to gastrointestinal disorders (nausea, vomiting and taste perversion), U.S. Pat. No. 4,842,866 assigned to Abbott Laboratories teaches a pharmaceutical dosage form comprising clarithromycin, a water-soluble alginate salt, and a water-insoluble complex salt of alginic acid. However, reproducibly bioavailable dosage forms could not be produced. In contrast, the incorporation of an organic carboxylic acid, such as citric acid (described in U.S. Pat. No. 5,705,190 assigned to Abbott), mainly to increase the bioavailability of the erythromycin derivative which is poorly soluble in intestinal fluids, resulted in once a day dosage forms bioequivalent to the marketed immediate release twice-daily compositions. However, these compositions exhibited high maximum plasma concentrations (Cₘₐₓ), thereby failing to minimize the adverse effects relating to gastrointestinal disorders including nausea, vomiting, and taste perversion. U.S. Pat. No. 6,010,718 assigned to Abbott discloses a method for preparing controlled release matrix tablet formulation (500 mg tablet weighing about 1000 mg) comprising from about 5% to about 50% by weight of a pharmaceutically acceptable polymer. The tablet formulations comprising 10, 20 and 30% by weight of low viscosity hydroxypropylmethylcellulose (Methocel™ K 100 LV from Dow Chemical Company) were observed to induce statistically significantly lower mean fluctuation index in the plasma than an immediate release composition of the erythromycin derivative while being substantially bioequivalent to the immediate release composition.

WO0149246 A2 (published 20010712) assigned to L. Oner and A. Toksoz discloses pharmaceutical compositions comprising from about 3% to about 40% by weight of hydrophilic hydroxyalkylcelluloses. US Patent Publication No. US2001094170 20010829 with the publication date of June 27^{th}, 2002 relates to a controlled release pharmaceutical composition comprising from about 0.1% to about 4% by weight of one or more pharmaceutically acceptable dissolution rate controlling polymers, such as carbohydrate gums, polyuronic acid salts, cellulosic ethers, acrylic acid polymers and mixtures thereof.

### SUMMARY OF THE INVENTION

Clarithromycin, a semi-synthetic macrolide antibiotic derived from erythromycin A, is a broad spectrum antibiotic. It is indicated for the treatment of a wide variety of respiratory and dermatological infections in children and adults. It is poorly water soluble. The present invention provides a method for preparing an extended release tablet dosage form of a poorly water soluble macrolide antibiotic for once a day oral administration as two 500 mg tablets or one 1000 mg tablet of convenient size/shape instead of two Biaxin XL, 500 mg, each tablet weighing about 1000 mg.

In accordance with one embodiment of the present invention, the poorly water soluble macrolide antibiotic drug-containing granulation is prepared in a granulation device, preferably a high shear granulator by first mixing milled or micronized clarithromycin and pharmaceutically acceptable hydrophilic excipients such as lactose, mannitol, microcrystalline cellulose and the like and adding an aqueous solution of a binder such as polyvinylpyrrolidone (Povidone K 29/32) in 0.005-0.05N hydrochloric acid and drying the granulation. Alternatively, the binder may be added in the dry form to the clarithromycin and excipient mass in the high shear granulator, and adding 0.005-0.05N hydrochloric acid to form granules. The dried granulation is milled, blended with suitable compression aids such as a diluent (for example, lactose, mannitol, microcrystalline cellulose and the like), a glidant (for example, colloidal silicon dioxide, talc and the like) and a lubricant (for example, magnesium stearate, stearic acid and the like), and compressed into clarithromycin ER (extended release) Tablets, 500 and 1000 mg with a drug release profile designed to provide appropriate plasma concentration profiles of the antibiotic over an extended period for the treatment of a wide variety of respiratory and dermatological infections in children and adults. By virtue of its small size and once a day dosing regimen, the dosage form prepared in accordance with the present invention should significantly improve patient compliance.

### BRIEF DESCRIPTION OF THE FIGURES

The invention will be described in further detail with reference to the accompanying Figures wherein:

FIG. 1 shows an in vitro drug release profile from 1000 mg clarithromycin extended release (ER) dosage form with different levels of lubricant and glidant, prepared according to Examples 1, 2 and 3 of the present invention in comparison to that of the commercially available clarithromycin extended release tablets, Biaxin XL, 500 mg, similarly dissolution tested. The tablets weigh approximately 1200 mg and contain no dissolution rate controlling polymers.

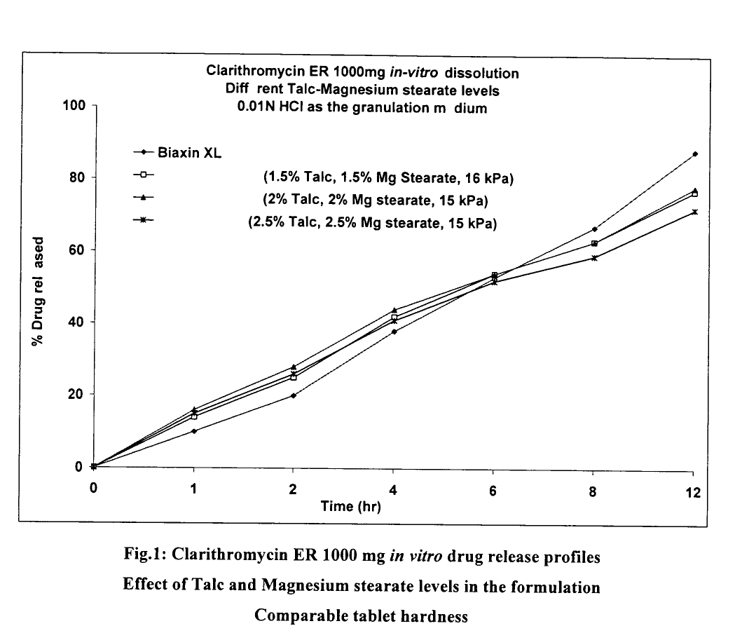

FIG. 2 is a comparison of the in vitro drug release profiles from 1000 mg clarithromycin ER tablets, weighing approximately 1200 mg, of Examples 1 and 4, prepared in the absence and presence of a tableting-aid agent, Microcrystalline cellulose, respectively. The drug release rate is not affected in the presence of the tabletting-aid agent; however, it was observed that the tableting process was significantly improved, thereby indicating the potential use of MCC.

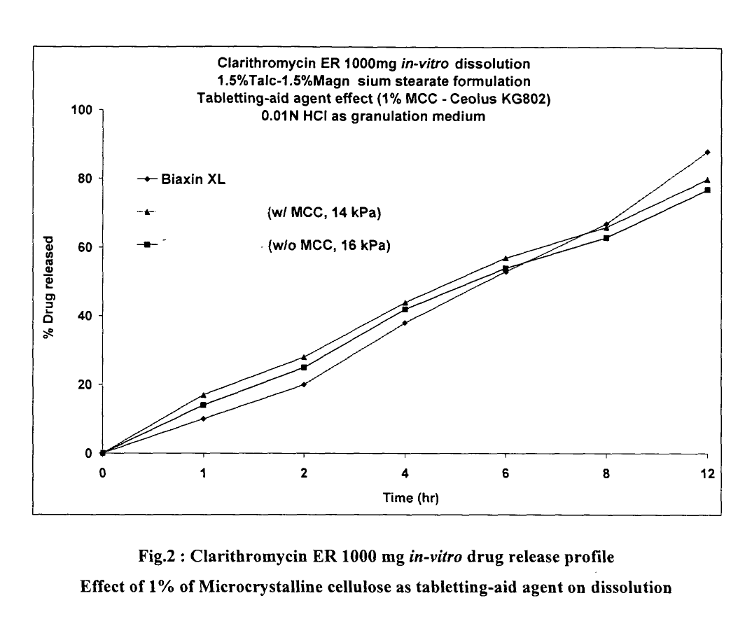

FIG. 3 shows a comparison of the in vitro drug release profile from 500 mg and 1000 mg clarithromycin ER tablets weighing approximately 600 mg and 1200 mg, respectively, and containing no dissolution rate controlling polymers, prepared according to Examples 2 and 5 of the present invention, in comparison to that of the commercially available clarithromycin ER tablets, Biaxin XL, 500 mg, similarly dissolution tested.

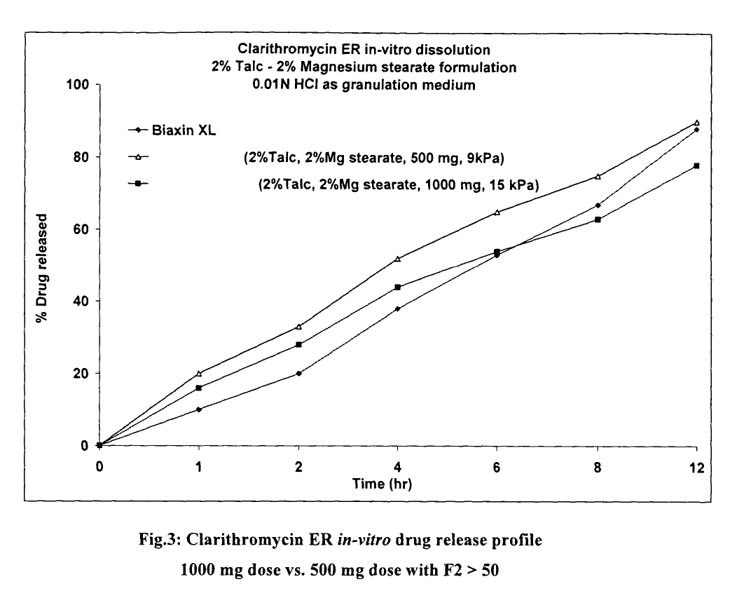

### DETAILED DESCRIPTION OF THE INVENTION

One embodiment of the present invention provides a method of making an extended release tablet dosage form comprising the steps of:
preparing a drug-containing granulation by mixing a macrolide antibiotic and a pharmaceutically acceptable diluent and granulating by (a) adding a solution of a binder optionally acidified with a mineral acid to form agglomerates, or (b) adding a dry binder and granulating by adding solution of an acid;
drying and milling to produce granulates of a suitable particle size distribution;
blending with a glidant, a lubricant, and optionally a filler and compressing into tablets of appropriate strength, hardness and friability.

The extended release (ER) tablets thus produced exhibit desired in vitro and in vivo drug release profiles, and are suitable for once-a-day oral administration in children and adults for the treatment of microbial infection.

Dissolution Procedure:

Drug release profiles of clarithromycin ER tablets can be determined according to the following procedure:
Dissolution testing is conducted with a USP Apparatus 2 (Paddles at 50 rpm, 900 mL of 0.1M sodium acetate buffer at pH 5.0 at 37° C.). Drug release with time is determined by HPLC on samples pulled at selected intervals.

The extended release (ER) tablets prepared in accordance with one embodiment of the present invention release the antibiotic in an amount not more than 35%, more preferably not more than 30%, and most preferably not more than 25% in 2 hours, about 30-60%, more preferably about 30-50%, and most preferably about 35-45% in 4 hours, about 50-90%, more preferably about 55-85%, and most preferably about 60-85% in 8 hours, and not less than 70%, more preferably not less than 80%, and most preferably not less than 85% in 12 hours.

An aqueous or a pharmaceutically acceptable solvent medium acidified with hydrochloric acid may be used for preparing granulations containing a macrolide antibiotic. A macrolide antibiotic, a film forming binder, and a pharmaceutically acceptable diluent may be blended together in a planetary mixer, a high shear granulator such as Fielder or a fluid bed granulator such as Glatt or a GMX Freund and granulated to form agglomerates by adding/spraying a granulating fluid containing a mineral acid. Alternatively, the binder may be added as a solution in the granulating fluid and the resultant mass granulated with an aqueous solution of the binder and a mineral acid. The wet mass may be milled, if required, suitably dried and is generally dry milled to produce granulate with the desired particle size distribution. In these embodiments, the drug load could be as high as 90% by weight of the granulation. The granulation is blended with a glidant, a lubricant, and optionally a filler and compressed into tablets of suitable strength using a tablet press.

Poorly water soluble macrolide antibiotics suitable for the preparation of ER tablets in accordance with the present investigation include erythromycin, clarithromycin, azithromycin and other erythromycin derivatives. The antibiotic is typically pre-milled to an average particle size of about 3 to 60 microns, more preferably about 3 to 20 microns, and most preferably about 3 to 15 microns.

The type of film forming binder that is used to bind the drug is usually a water soluble, alcohol soluble or acetone/water soluble binder. Binders such as polyvinylpyrrolidone (PVP), average viscosity of 3 to 15 cps hydroxypropyl methylcellulose (HPMC), average viscosity of 3 to 15 cps hydroxypropylcellulose (HPC known by the trade name of Klucel® LF), dextran, and corn starch may be used at concentrations of about 0.5 to 5 weight %. The binder is preferably a hydrophilic binder such as polyvinylpyrrolidone and used for granulation in an amount of about 1% to about 4% w/w based on the total tablet weight, added either in the dry form or as an aqueous solution of a mineral acid.

The mineral acids suitable for improving in vitro/in vivo drug release profiles for improved bioavailability from extended release tablet formulations include hydrochloric acid and sulfuric acid, though hydrochloric acid is preferred. The mineral acid is used in a concentration of about 0.0005 to 0.05N.

Typical pharmaceutically acceptable diluents (tableting aid) suitable for preparing ER tablets of poorly water soluble macrolide antibiotics in accordance with the present invention include lactose, mannitol, microcrystalline cellulose, calcium sulfate, potassium dihydrogen phosphate and the like. These materials are typically used in an amount of about 1 to 5_% w/w individually or in combination thereof. The pharmaceutically acceptable diluent in the granulation includes lactose at a concentration of from about 5% to about 35% w/w, preferably from about 7.5% to about 17.5% w/w based on the total tablet weight.

Representative examples of lubricants useful in the invention include magnesium stearate, talc, calcium stearate, stearic acid and mixtures thereof. The preferred glidant useful in the invention is colloidal silicon dioxide, commercially available as Cab-O-Si™. The lubricants are typically used in an amount of about 2 to 8_% w/w. The glidants are typically used in an amount of about _{—}0.1_to_{—}0.5% w/w.

The tablets of the present invention may be film coated for aesthetic reasons or for providing a moisture barrier. A hydroxypropyl methylcellulose (Opadry™) film using any of the coating techniques commonly used in the pharmaceutical industry may be applied, but pan coating is particularly useful. While HPMC is typically used, other barrier coating materials, such as hydroxypropylcellulose (HPC) or sugar can also be used. These films are typically applied in an amount of about 1 to 4% w/w based on the total weight of the coated tablet.

Thus, the pharmaceutically acceptable excipients suitable for preparing ER tablets of poorly water soluble macrolide antibiotics in accordance with the present invention can be selected from conventional excipients widely used in the preparation of immediate release dosage forms. None of these excipients is a dissolution rate controlling polymer such as an ester of a fatty acid or a wax conventionally used in the preparation of controlled release dosage forms.

The tablet is typically prepared by (1) granulating the antibiotic, e.g., clarithromycin, at a concentration of from about 62% to about 90% w/w based on the total tablet weight with a pharmaceutically acceptable filler such as lactose, mannitol, microcrystalline cellulose and the like, using an aqueous solution of a hydrophilic binder which is optionally acidified with hydrochloric acid to a normality ranging from about 0.005 to about 0.05, (2) blending said granules with tableting aids such as magnesium stearate, fine colloidal silicon dioxide, talc, microcrystalline cellulose and/or lactose, and (3) compressing the blend into 500 mg or 1000 mg tablets in the weight range of about 575-750 mg and 1120-1500 mg, respectively. The granulation is typically dry milled to pass through at lease a # 20 mesh sieve to obtain a free flowing granulation suitable for compression on a high speed tablet press. The compression pressure used to prepare tablets is not critical as long as tablets with acceptable hardness (e.g., not less than 10 kP for 1000 mg tablets) and friability (e.g., not more than 1%, preferably not more than 0.5% for 1000 mg tablets) are produced to meet all of the industrial applicability criteria, namely, product quality, suitability for packaging in HDPE bottles and blisters for storage, transportation, commercial distribution, and use, including exhibiting the drug release profiles similar to that described above.

The following non-limiting examples illustrate the Clarithromycin ER dosage forms manufactured in accordance with the invention, which exhibit in vitro drug release profiles, similar to that of the commercially available Clarithromycin ER tablets, Biaxin XL, 500 mg weighing approximately 1000 mg, prepared in accordance with U.S. Pat. No. 6,010,718 assigned to Abbott, comprising a significant fraction of a dissolution rate controlling polymer (s).

### EXAMPLE 1

84.75 parts of Clarithromycin were granulated in a high shear granulator with 10.05 parts of lactose monohydrate using 2.0 parts of PVP K29/32 which is prepared in an aqueous solution of hydrochloric acid with a final normality of 0.01N. The granulation was wet milled, dried in a Metro oven for an LOD (loss on drying) of less than 2%, and dry milled to have a particle distribution of less than 600 microns. The milled granulation was blended with 1.50 parts of magnesium stearate, and 1.50 parts of Talc and compressed into 1000 mg Extended Release Clarithromycin tablets weighing about 1200 mg.

### EXAMPLE 2

83.75 parts of Clarithromycin were granulated in a high shear granulator with 10.02 parts of lactose monohydrate using 2.0 parts of PVP K29/32 which is prepared in an aqueous solution of hydrochloric acid with a final normality of 0.01N. The granulation was wet milled, dried in a Metro oven for an LOD (loss on drying) of less than 2%, and dry milled to have a particle distribution of less than 600 microns. The milled granulation was blended with 2.01 parts of magnesium stearate, and 2.01 parts of talc in a V-blender and compressed into 1000 mg ER Clarithromycin tablets weighing approximately 1200 mg.

### EXAMPLE 3

82.92 parts of Clarithromycin were granulated in a high shear granulator with 9.92 parts of lactose monohydrate using an aqueous solution of 2.0 parts of polyvinylpyrrolidone (Povidone K 29/32, a binder) acidified with hydrochloric acid for a final normality of 0.01N. The granulation was wet milled, dried in a Metro oven for an LOD (loss on drying) of less than 2%, and dry milled to have a particle distribution of less than 600 microns. The milled granulation was blended with 2.5 parts of magnesium stearate, and 2.5 parts of talc in a V-blender and compressed into 1000 mg ER Clarithromycin tablets weighing approximately 1200 mg.

### EXAMPLE 4

83.89 parts of Clarithromycin were granulated in a high shear granulator with 9.95 parts of lactose monohydrate using an aqueous solution of 2.0 parts of polyvinylpyrrolidone (Povidone K 29/32, a binder) acidified with hydrochloric acid for a final normality of 0.01N. The granulation was wet milled, dried in a Metro oven for an LOD (loss on drying) of less than 2%, and dry milled to have a particle distribution of less than 600 microns. The milled granulation was blended with 1.01 parts of microcrystalline cellulose, 1.48 parts of magnesium stearate, and 1.48 parts of Talc in a V-blender and compressed into 1000 mg Extended Release Clarithromycin tablets weighing approximately 1200 mg.

### EXAMPLE 5

83.76 parts of Clarithromycin were granulated in a high shear granulator with 10.02 parts of lactose monohydrate using an aqueous solution of 2.0 parts of polyvinylpyrrolidone (Povidone K 29/32, a binder) acidified with hydrochloric acid for a final normality of 0.01N. The granulation was wet milled, dried in a Metro oven for an LOD (loss on drying) of less than 2%, and dry milled to have a particle distribution of less than 600 microns. The milled granulation was blended with 2.01 parts of magnesium stearate, and 2.01 parts of Talc in a V-blender and compressed into 500 mg Extended Release Clarithromycin tablets weighing approximately 600 mg.

Having described the invention in detail and by reference to specific embodiments thereof, it will be apparent the numerous modifications and variations are possible without departing from the spirit and scope of the invention.

1. An oral solid pharmaceutical dosage form comprising an extended release (ER) tablet, wherein said ER tablet comprises:
a. an effective amount of a macrolide antibiotic selected from clarithromycin, azithromycin, or an erythromycin derivative;
b. from about 2% to about 40% w/w of one or more pharmaceutically acceptable water soluble excipients;
c. one or more tableting aids;
d. wherein the dosage form does not contain a dissolution rate controlling polymer; and
e. wherein said tablet when tested in a USP Apparatus 2 at 50 rpm using 900 mL 0.1M sodium acetate buffer (pH=5.0) at 37° C. exhibits a dissolution profile substantially corresponding to the following pattern:
not more than 35% of the total antibiotic is released in 2 hours;
about 30-60% of the total antibiotic is released in 4 hours;
about 50-90% of the total antibiotic is released in 8 hours; and
not less than 70% of the total antibiotic is released in 12 hours.

2. A pharmaceutical dosage form as defined in claim 1, wherein said dissolution profile substantially corresponds to the following pattern:
not more than 30% of the total antibiotic is released in 2 hours;
about 30-50% of the total antibiotic is released in 4 hours;
about 60-85% of the total antibiotic is released in 8 hours; and
not less than 85% of the total antibiotic is released in 12 hours.

3. A pharmaceutical dosage form as defined in claim 2, wherein said tablet core is prepared by (1) granulating clarithromycin, at a concentration of from about 62% to about 90% w/w based on the total tablet weight with a pharmaceutically acceptable filler selected from the group consisting of lactose, mannitol, and microcrystalline cellulose, using an aqueous solution of a hydrophilic binder which is optionally acidified with hydrochloric acid to a normality ranging from about 0.005 to about 0.05, (2) blending said granules with a tableting aid selected from the group consisting of magnesium stearate, fine colloidal silicon dioxide, talc, microcrystalline cellulose and/or lactose, and (3) compressing the blend into 500 mg or 1000 mg tablets in the weight range of about 575-750 mg and 1120-1500 mg, respectively.

4. A pharmaceutical dosage form as defined in claim 1 wherein the pharmaceutically acceptable diluent in the granulation includes lactose at a concentration of from about 5% to about 35% w/w.

5. A pharmaceutical dosage form as defined in claim 4 wherein said hydrophilic binder is selected from the group consisting of polyvinylpyrrolidone, hydroxypropylmethylcellulose, hydroxypropylcellulose, corn, starch, and dextran for granulation at a concentration of from about 1% to about 4% w/w based on the total tablet weight, added either in the dry form or as an aqueous solution of a mineral acid.

6. A pharmaceutical dosage form as defined in claim 3 wherein said tableting aid includes lactose blended at a concentration of about 1-5% by weight of total tablet weight.

7. A pharmaceutical dosage form as defined in claim 3 wherein said tableting aid includes microcrystalline cellulose blended at a concentration of about 1-4% by weight for a total polymer content of not more than 5% by weight of the tablet.

8. A pharmaceutical dosage form as defined in claims 3 wherein said tableting aid is magnesium stearate alone or in combination with talc externally blended at a total concentration of from about 1.0% to about 10% by weight.

9. A pharmaceutical dosage form as defined in claims 1-3 wherein said tableting aid is colloidal silicon dioxide externally blended at a concentration of about 0.1-0.5% by weight of total tablet weight.

10. A production method for the preparation of an extended release clarithromycin tablet, comprising the steps of:
a. preparing a granulation of clarithromycin in a high shear granulator, comprising a pharmaceutically acceptable filler/diluent and an aqueous solution of a binder, optionally acidified using HCl for a normality of 0.005-0.05;
b. blending said granulation with other non-dissolution rate controlling excipients selected from the group consisting of a glidant, talc, a filler, and a lubricant;
c. compressing the blend to produce 500 mg or 1000 mg extended release clarithromycin tablets using a rotary tablet press;
wherein the tablet exhibits a dissolution profile that substantially corresponds to the following pattern:
not more than 35%, of the total clarithromycin is released in 2 hours;
about 30-60%, of the total clarithromycin is released in 4 hours;
about 50-90%, of the total clarithromycin is released in 8 hours; and
not less than 70%, of the total clarithromycin is released in 12 hours.

11. The method of claim 10 wherein said clarithromycin-containing tablet core may be provided with a film coat.

12. The method of claim 10 wherein said dissolution profile substantially corresponds to the following pattern:
not more than about 30% of the total clarithromycin is released in 2 hours;
about 30-50% of the total clarithromycin is released in 4 hours;
about 60-85% of the total clarithromycin is released in 8 hours; and
not less than about 85% of the total clarithromycin is released in 12 hours.
